(19) United States
(12) Patent Application Publication
(10) Pub. No.: US 2021/0196379 A1
(43) Pub. Date: Jul. 1, 2021

(54) STAND-OFF CATHETER WITH MULTIPLE LASER-FIRING PORTS

(71) Applicant: Optical Integrity, Inc., Panama City Beach, FL (US)

(72) Inventors: JOE DENTON BROWN, PANAMA CITY BEACH, FL (US); DANIEL MALPHURS, PANAMA CITY BEACH, FL (US)

(21) Appl. No.: 17/135,225

(22) Filed: Dec. 28, 2020

Related U.S. Application Data
(60) Provisional application No. 62/954,005, filed on Dec. 27, 2019.

(51) Int. Cl. A61B 18/24

(52) U.S. Cl. CPC A61B 18/24; A61B 2018/225

(57) ABSTRACT

A surgical laser delivery systems utilizes a standoff catheter to prevent contact and/or maintain a predetermined spacing between the laser delivery fiber or fiber assembly and the target. The standoff catheter includes multiple side-firing ports. The side-firing ports may include openings of the same or different dimensions, at least one diffuser, and/or at least one lens, to permit the operator to control the degree of vaporization/coagulation and/or vary lasing parameters without having to withdraw the optical fiber from the patient, by rotating or linearly moving the laser or laser assembly with respect the standoff catheter. The side-firing ports may also include an opening at the front of the stand-off catheter to enable use with a forward-firing laser fiber or fiber assembly.

This application claims the benefit of U.S. Provisional Patent Appl. Ser. No. 62/954,005, filed Dec. 27, 2019.

### BACKGROUND OF THE INVENTION

### 1. Field of the Invention

The invention relates to field of laser surgery, and in particular to systems and devices for delivering radiation to a treatment target for purposes of ablation, vaporization, and/or coagulation during a variety of surgical procedures. The procedures may include, but are not limited to, varicose vein ablation, benign prostate hyperplasia (BPH) treatment, and kidney stone removal.

More particularly, the invention involves laser delivery systems and devices that utilize a standoff catheter to prevent contact and/or maintain a predetermined spacing between the laser delivery fiber or fiber assembly and the target, in order to reduce fiber degradation and allow extended use or reuse of the fiber. The standoff catheter may be arranged to accommodate a side-firing laser delivery fiber or fiber assembly or, according to one aspect of the invention, both side-firing and forward-firing laser delivery fiber or fiber assemblies.

When arranged to accommodate a side-firing laser delivery fiber or fiber assembly, the standoff catheter may include both an irrigation opening at the front end of the catheter, through which irrigating fluid flows, and openings through which laser energy is passed. The openings for laser energy preferably have a diameter that is close to a diameter of a laser beam, so as to prevent ingress of debris and in which an air bubble may be formed to reduce attenuation of the laser beam.

The side-firing ports may include multiple openings of the same or different dimensions, at least one diffuser, and/or at least one lens, to permit the operator to control the degree of tissue vaporization/coagulation by the laser beam, and/or vary lasing parameters, without having to withdraw the optical fiber from the patient, by rotating or linearly moving the laser or laser assembly with respect the standoff catheter.

Optionally, the side openings may initially be provided as “knock outs,” i.e., pre-scored or partially drilled removable sections that leave openings when knocked or punched out when a laser is fired at the knock out. In addition, the irrigation opening at the front of the stand-off catheter may by arranged to enable use of the standoff catheter with a forward-firing laser fiber or fiber assembly as well as with a side-firing laser fiber.

### 1. Description of Related Art

A variety of devices and methods have previously been proposed to prevent contact between tissues or debris during surgical laser procedures involving tissue vaporization or coagulation, or use of the laser to destroy objects such as stones. Depending on the surgical procedure, surgical laser systems can utilize either side firing laser fibers or forward firing laser fibers. Each type of procedure may be subject to fiber wear or damage, necessitating relative frequent fiber replacement, and other problems that may affect treatment efficacy and possibly pose risks to a patient. For example, many surgical laser procedures produce debris, which can accumulate on the fiber and cause degradation of the fiber. In addition, the need for an operator to position the fiber in close proximity to the fiber will frequently result in at least momentary contact between the tissue and fiber, which enhances fiber erosion and can cause flashes that interfere with the operator's view of the treatment site and fiber position.

a. Side-Firing Laser Fibers

For side-firing laser fibers, protection of the fiber has conventionally involved use of a transparent cap through which the laser is fired and that is permanently affixed to the fiber to form a laser delivery fiber assembly. The side-firing fiber assembly may be inserted into or combined with an external tube or sleeve that enables flow of irrigation fluid past the fiber tip, in order to cool and flush debris away from the fiber. An early version, developed during the 1990s, included a transparent quartz or dielectric cap fitted over the end of a laser having an angled tip, the cap including a front facing opening for passage of irrigation fluid, with the angled tip causing the laser to be fired in a generally radial direction through the cap. Because surgical lasers at the time used relatively low power green laser light, the laser light passed through the irrigation fluid without vaporization. The principles developed for the side-firing laser systems of the 1990s are still utilized in current commercially-available side-firing surgical laser systems, such as the Moxy™ system described in U.S. Pat. No. 8,858,542, even though the frequency or power of lasers has increased.

A disadvantage of the Moxy™ system is that the permanently affixed transparent cap will accumulate debris, causing degradation of the cap and increasing attenuation of the laser as it is fired through the cap. In order to prevent excessive debris accumulation, frequent cleaning of the cap and/or frequent replacement of the entire fiber/cap arrangement is necessary, which results in high cost and extended treatment times.

An alternative to the Moxy™ system is the system disclosed in U.S. Pat. No. 6,802,838 (the Trimedyne™ system), which replaces the Moxy™ cap and sleeve with a catheter that is crimped to the fiber or secured thereto by an insert, and that includes a window through which the laser is fired, as well as multiple ports for fluid input and debris removal. Because the catheter is fixed to the fiber, the Trimedyne™ system suffers at least the same cost and treatment-time disadvantages as the Moxy™ system. In addition, the Trimedyne™ system as the additional disadvantage of providing only a single window for emission of the radiation, with the result that degradation caused by the laser is concentrated in the area of the window.

Copending U.S. patent application Ser. No. 15/234,455, filed Aug. 11, 2016 and incorporated herein by reference, overcomes the disadvantages of both the Moxy™ and Trimedyne™ systems by replacing the fixed cap or catheter with a disposable or removable sleeve that is fitted over the side firing laser fiber and/or the Moxy™ outer sleeve. This improvement enables debris accumulation or damage to the outer tube to be addressed by simply removing and replacing the sleeve, without the need to interrupt a surgical procedure replace the fiber itself, or to clean the fiber, Moxy™ cap, or Trimedyne™ catheter.

Copending U.S. patent application Ser. No. 15/957,085, filed Apr. 19, 2018, and also incorporated by reference herein, discloses further variations of conventional side-firing laser systems such the Moxy™ and Trimedyne™ catheters or sleeves, as well as variations of the replaceable catheters or sleeves disclosed in U.S. patent application Ser. No. 15/234,455. These further variations include designs in which the outer sleeve is in the form of a catheter that, unlike the catheter of the Trimedyne™ system, is modified so that it can be easily removed and replaced without having to also dispose of the fiber, and which includes a firing window that extends circumferentially around the catheter to enable the fiber to be rotated within the catheter in order to limit damage caused by firing of the laser to any one area of the catheter.

b. Forward-Firing Laser Fibers

Standoff catheters can also be used in connection with forward firing laser fibers. For example, copending U.S. patent application Ser. No. 15/992,609, filed May 30, 2018; U.S. patent application Ser. No. 16/234,690, filed Dec. 28, 2018; and U.S. patent application Ser. No. 16/353,225, filed Mar. 14, 2019, all disclose forward firing arrangements in which the catheter serves to space the tip of the laser from an object to be lased. Each of these copending patent applications is incorporated by reference herein, and each shows arrangements are especially suitable for lithotripsy procedures, in which use of the catheter has the additional advantage of providing a cavitation effect that decreases attenuation of the laser beam and thereby provides enhanced efficiency and decreased treatment time.

The standoff catheters disclosed in the copending applications include embodiments in which the sleeve is disposable or replaceable, allowing the sleeves to be replaced without having to replace the more expensive fiber. Not only does this provide a simple and inexpensive way to extend fiber life and avoid the need for time-consuming fiber replacement during a surgical procedure, but it provides a degree of modularity, allowing interchangeability of catheter tips. Nevertheless, even though replacement of a catheter does not require a great deal of time, any interruption will increase the duration of the procedure and therefore risk to the patient. Also, it would be advantageous to provide a single catheter design that can accommodate a variety of lasing configurations or procedures, without the need to provide a different catheter for each procedure.

### SUMMARY OF THE INVENTION

It is an objective of the invention to provide standoff catheters having enhanced versatility, durability, and/or performance.

It is a further objective of the invention to provide standoff catheters having variable configurations, or that can be used for different surgical laser procedures.

One or more of these objectives is achieved, in various exemplary embodiments of the invention, by providing a standoff catheter having multiple openings through which a laser may be fired, the openings being separate from and in addition to any irrigation opening. In these embodiments, the opening preferably has a diameter corresponding to or smaller than the diameter of a laser to be fired through the opening, so that the laser vaporizes any fluid in the opening and forms a bubble through which the laser beam passes with minimal attenuation. The term “diameter of the beam” in this context may, for example, refer to the Gaussian beam radius (1/e² value). The openings may correspond to the opening described in may generally correspond to the radially-extending lasing opening or aperture described in the above-cited U.S. patent application Ser. No. 15/957,085, filed Apr. 19, 2018, which has a size that is too small for a substantial amount of a liquid such as irrigating fluid to pass, but which is just large enough to permit passage of most or all of the laser beam.

In a preferred embodiment of the invention, the standoff catheter includes multiple openings arranged on a side surface of the catheter, the openings and surgical laser fiber being relatively movable to an initial lasing position in which a first opening is in the path of the laser so that the laser beam can be fired through the opening, and subsequently relatively movable to a different lasing position in which a second opening is in the path of the laser. In this embodiment, the first, second, and/or additional openings can have the same size, different sizes, or combinations of same and different sizes. By providing openings of the same size, the operational life of the standoff catheter can be increased by allowing lasing through the second opening when the first opening has degraded. On the other hand, by providing openings of a different size, the standoff catheter can be used for different procedures and/or beam dimensions.

The openings may be through-holes whose dimensions are selected so that, as the laser beam passes through the opening, fluid in the opening is vaporized to enhance lasing efficiency. In addition, the catheter may also serve as Moxy™-type sleeve to permit passages of irrigation fluid, and include a front opening that permits passage of the irrigation fluid.

Alternatively, one or more of the openings may be replaced by at least one diffuser and/or at least one lens to attenuate, focus, collimate, or direct the laser light for procedures where the intensity of area over which the energy is applied needs to be varied. The diffusers and lenses can also be identical or varying properties, or the openings may include a combination of through-holes and diffusers or lenses that permit the intensity to be varied by moving the catheter from a position in which a through-hole is positioned in the path of the laser to a position in which a selected one of the diffusers or lenses is positioned in the path of the laser.

Also as an alternative, the openings may initially be provided in the form of knock outs, partially drilled, or scored sections that are removed or that pop out when a laser is fired at them.

According to yet another aspect of the preferred embodiment, the catheter may be moved linearly or by rotation, with the openings arranged along the axis of the fiber for linear movement and around a circumference of the fiber for rotational movement.

### BRIEF DESCRIPTION OF THE DRAWINGS

FIGS. 1-3 are isometric views showing variations of a first preferred embodiment of the invention.

FIG. 4 includes cross-sectional side views of a stand-off catheter according to the first preferred embodiment, into which a side-firing laser assembly and a forward-firing laser assembly have been respectively inserted.

FIG. 5 is an isometric view of a second preferred embodiment of the invention.

### DETAILED DESCRIPTION OF THE PREFERRED EMBODIMENTS

Throughout the following description and drawings, like reference numbers/characters refer to like elements. It should be understood that, although specific exemplary embodiments are discussed herein there is no intent to limit the scope of present invention to such embodiments. To the contrary, it should be understood that the exemplary embodiments discussed herein are for illustrative purposes, and that modified and alternative embodiments may be implemented without departing from the scope of the present invention.

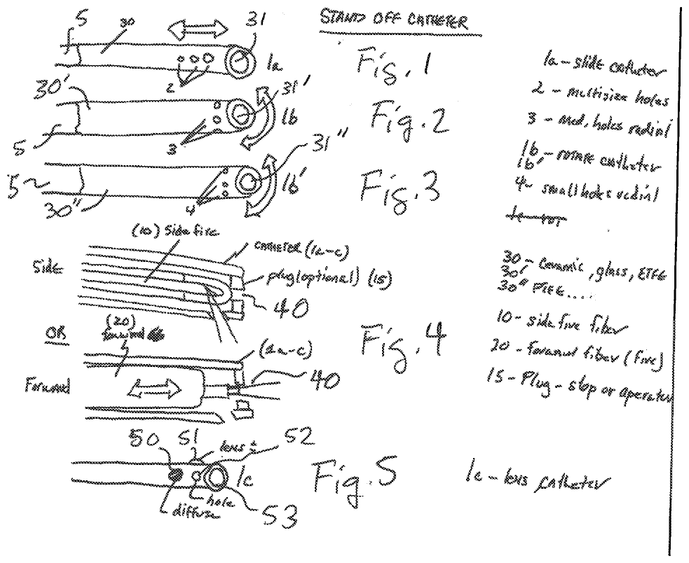

FIGS. 1-3 show stand-off catheters 1a, 1b, and 1b′, each having a cylindrical main body 30, 30′, and 30″ made of ceramic, glass, ETFE, PTFE, etc. that is coupled or affixed to, integral with, part of a catheter, introducer or sleeve 5 through which an optical fiber or optical fiber assembly is inserted. The main bodies 30, 30′, 30″ include respective openings or apertures 2, 3, or 4 that may generally correspond to the radially-extending lasing opening or aperture described in the above-cited U.S. patent application Ser. No. 15/957,085, filed Apr. 19, 2018, which has a size that is too small for a substantial amount of a liquid such as irrigating fluid to pass, but which is just large enough to permit passage of most or all of the laser beam. Such an opening or aperture has the advantage that any liquid present in the path of the beam will be vaporized or turned to steam, forming an air channel from the fiber to the tissue in order to minimize absorption of the laser beam, and therefore optimizing the amount of energy delivered to the tissue for therapeutic purposes.

Since the size of the opening depends on the cross-sectional dimensions of the beam, i.e., on the beam width, diameter, or Gaussian beam radius (1/e² value), a single opening can only be used with a beam having particular dimensions. According to the present invention, however, the stand-off catheter can be modified for use with beams having different dimensions, by providing an array or series of the openings or apertures 2, 3, or 4 of different sizes. Larger beams, for example, will deliver therapeutic radiation over a larger area of tissue, while narrower beams permit more precise energy delivery and/or delivery of radiation having a higher intensity.

As shown in FIGS. 1-3, the openings 2, 3, and 4 can be arranged linearly or circumferentially around the catheter, and may include multiple apertures or openings or each size, so that the laser can be positioned relative to a desired opening side by either linearly moving the lasing tip of the optical fiber, and/or by rotating the tip. It is also possible for all of the openings or apertures on a single catheter to have the same dimensions, so that when one opening has degraded as a result of lasing, the laser can be positioned to lase through another non-degraded opening without having to replace the catheter.

Optionally, each of the openings may be formed as a knock out, pre-drilled, or pre-scored section that forms a temporary plug and pops out when exposed to a laser beam. When one opening is used and excessively widened or drilled out by passage of the laser, the laser can be moved to the location of another knock out, which pops out to form an opening of a desired size so that the surgical procedure can continue without undue interruption.

The distal ends 30, 30′, 30″ of the catheters of FIGS. 1-3 are preferably open to permit passage of irrigation fluid. In addition, providing open-ended catheters enables each catheter to also be used as a stand-off catheter for forward-firing laser systems, as described by way of example and not limitation in the above-cited copending U.S. patent application Ser. Nos. 15/992,609; 16/234,690; and 16/353,225.

As illustrated in FIG. 4, the front opening 40 may have a diameter that is narrower than the inner diameter of the remainder of the catheter in order to form a shoulder and limit forward movement of, or facilitate positioning of, a side-firing laser assembly, and also to control the size of the passage when used with a forward-firing laser. When used with a forward-firing laser, an air bubble can be formed in the front opening of passage during lasing to increase energy transfer efficiency. As an example, any of the catheters 1a, 1b, 1b′, and 1c of FIGS. 1-3 and 5 may be provided with a plug having a desired opening size to form the forward-firing lasing passage, with the plugs further being changeable to control the opening size.

In the embodiment shown in FIG. 5, the catheter 1 c is further provided with at least one diffuser 50 and/or at least one lens 51, which are in addition to aperture 52 corresponding to the one or more openings or apertures 2-4 shown in FIGS. 1-3 and optional front opening 53. The diffuser 50 can be used to intentionally attenuate or diffuse a side-firing laser beam for purposes such as coagulation, allowing the user to switch between a vaporization operation to a coagulation operation by moving the angled tip of the side-firing laser from a position adjacent one of the openings or apertures 52 to a position adjacent the at least one diffuser. On the other hand, the lens 51 can be used to focus, disperse, collimate or direct the laser beam as necessary for the particular procedure in question. If more than one diffuser or lens is provided, further control of the laser beam intensity, direction, and treatment area can be obtained.

Although the term “catheter” is used herein, it will be appreciated that the catheter may be referred to as an introducer or sleeve, that it is a cylindrical or generally cylindrical structure, and that the catheter may be inserted into the patient directly or via a laser endoscope, cystoscope, or resectoscope. In addition, it will be appreciated by those skilled in the art that the optical fiber, whether in the side or forward-firing configuration, my be included in an optical fiber assembly or be provided with a cap or ferrule through which the laser is fired. Such a cap or ferrule may further be wholly or partly made of a laser-transparent material, or include an opening to permit passage of the laser.

What is claimed is:

1. A stand-off catheter, comprising:
a cylindrical or generally cylindrical main body through which an optical fiber or fiber assembly is inserted to perform a surgical laser procedure,
wherein the main body includes at least two side openings, each configured to permit passage of a laser beam when the optical fiber or fiber assembly is a side-firing surgical laser fiber or fiber assembly selectively positioned to fire through a respective one of the at least two side openings.

2. A stand-off catheter as claimed in claim 1, wherein the at least two side openings are arrayed in a direction parallel to a cylinder axis of the main body.

3. A stand-off catheter as claimed in claim 1, wherein the at least two side openings are arranged circumferentially around the main body.

4. A stand-off catheter as claimed in claim 1, wherein the at least two side openings have mutually different dimensions.

5. A stand-off catheter as claimed in claim 1, wherein at least two of the side openings have same dimensions.

6. A stand-off catheter as claimed in claim 1, wherein the side openings are formed as pre-drilled sections that pop out when the laser beam is fired at a respective section during the surgical laser procedure.

7. A stand-off catheter as claimed in claim 1, wherein an end of the stand-off catheter is open to permit passage of a laser beam from a forward-firing laser.

8. A stand-off catheter as claimed in claim 7, wherein the open end of the stand-off catheter has a diameter that is smaller than an inner diameter of the main body to form a narrowed opening and a shoulder that limits movement of the optical fiber or optical fiber assembly in the forward direction.

9. A stand-off catheter as claimed in claim 8, wherein the narrowed opening is formed by a plug that fits into the front end of the stand-off catheter.

10. A stand-off catheter, comprising:
a cylindrical or generally cylindrical main body through which an optical fiber or fiber assembly is inserted to perform a surgical laser procedure,
wherein the main body includes at least one side opening and at least one diffuser or lens, each configured to permit passage of a laser beam when the optical fiber or fiber assembly is a side-firing surgical laser fiber or fiber assembly selectively positioned to fire through a respective one of the at least one side opening and at least one diffuser or lens.

11. A stand-off catheter as claimed in claim 10, wherein the at least one side opening and at least one diffuser or lens are arrayed in a direction parallel to a cylinder axis of the main body.

12. A stand-off catheter as claimed in claim 10, wherein the at least one side opening and at least one diffuser or lens are arranged circumferentially around the main body.

13. A stand-off catheter as claimed in claim 10, wherein the at least one side opening is formed as a pre-drilled section that pops out when the laser beam is fired at the section during the surgical laser procedure.

14. A stand-off catheter as claimed in claim 10, wherein an end of the stand-off catheter is open to permit passage of a laser beam from a forward-firing laser.

15. A stand-off catheter as claimed in claim 14, wherein the open end of the stand-off catheter has a diameter that is smaller than an inner diameter of the main body to form a narrowed opening and a shoulder that limits movement of the optical fiber or optical fiber assembly in the forward direction.

16. A stand-off catheter as claimed in claim 15, wherein the narrowed opening is formed by a plug that fits into the front end of the stand-off catheter.
